(19) United States
(12) Plant Patent Application Publication
(10) Pub. No.: US 2003/0024022 P1
(43) Pub. Date: Jan. 30, 2003

(54) Magnolia plant named 'Strgra'

(76) Inventors: Edward V. Strauss, Silky Oaks (AU); Patricia A. Strauss, Silky Oaks (AU)

Correspondence Address: FOLEY & LARDNER, Suite 500, 3000 K Street, N.W., Washington, DC, 20007-5109, US

(21) Appl. No.: 09/874,300

(22) Filed: Jun. 6, 2001

(51) Int. Cl. A01H 5/00

(52) U.S. Cl. PLT/223

(57) ABSTRACT

A new and distinct cultivar of magnolia named ‘STRgra’ which is smaller in size than its maternal parent, has an early flowering habit, deep green glossy leaves RHS 147A, 6 month flowering period, and strong sweet lemony scent. The cultivar has a larger growth habit than the closest comparative cultivar Magnolia grandiflora ‘Little Gem’.

### BACKGROUND OF THE INVENTION

The present invention comprises a new and distinct cultivar of Magnolia grandiflora, hereinafter referred to by the cultivar name ‘STRgra’ and having the common name Bull Bay Magnolia.

The new cultivar was isolated by the inventors at Uki, New South Wales, Australia, by seedling selection following self-pollination of the maternal parent, an unnamed plant of Magnolia grandiflora. The new cultivar was selected by the inventors, Edward V. Strauss and Patricia A. Strauss, primarily for its small size and early flowering habit. The new cultivar is distinguished from the maternal parent by its medium leaf size, small spreading growth habit, early flowering and medium flower size.

Following selection of the seedling, the cultivar was grown from cuttings propagated vegetatively by grafting. Following several generations of propagated cuttings, uniformity and stability of the selected characteristics was observed. Horticultural examination of the instant plant has demonstrated that the combination of characteristics herein disclosed for ‘STRgra’ are firmly fixed and are retained through successive generations of asexual reproduction.

The selected seedling was observed to flower in its second year, and was very florific over a long flowering season with smaller leaves and a small multi-branched plant habit about one third of the size of its sister seedlings. The following table of characteristics indicate the manner in which the new cultivar is distinct,

| | Leaf | | Early | Flower | Underside of |
|---|---|---|---|---|---|
| | Size | Tree Shape | Flowering | Size | Leaf |
| Maternal | Large | Tall | No | Large | Light |
| Parent: | | Spreading | | | Variable |
| Magnolia | | | | | |
| grandifora | | | | | |
| Comparative | Small | Medium | Yes | Small | Heavy |
| Cultivar: | | Upright | | | |
| Magnolia | | | | | |
| grandiflora | | | | | |
| “Little | | | | | |
| Gem” | | | | | |
| Present | Medium | Small | Yes | Medium | Light |
| Cultivar: | | Spreading | | | |
| Magnolia | | | | | |
| grandiflora | | | | | |
| ‘STRgra’ | | | | | |

### BRIEF DESCRIPTION OF THE INVENTION

The following traits have been repeatedly observed and are determined to be basic characteristics of ‘STRgra’ which, in combination, distinguish this Magnolia as a new and distinct cultivar:

1. early flowering habit;

2. deep, green glossy leaves, RHS 147A;

3. 6 month flowering period; and

4. strong, sweet, lemony scent.

‘STRgra’ has not been observed under all possible environmental conditions.

The phenotype of the new cultivar may vary significantly with variations in environment such as temperature, light intensity, and day length without any change in genotype. The following observations, measurements, and values describe the new cultivar as grown in Uki, New South Wales, Australia under conditions which approximate those generally used in commercial practice.

### BRIEF DESCRIPTION OF THE DRAWINGS

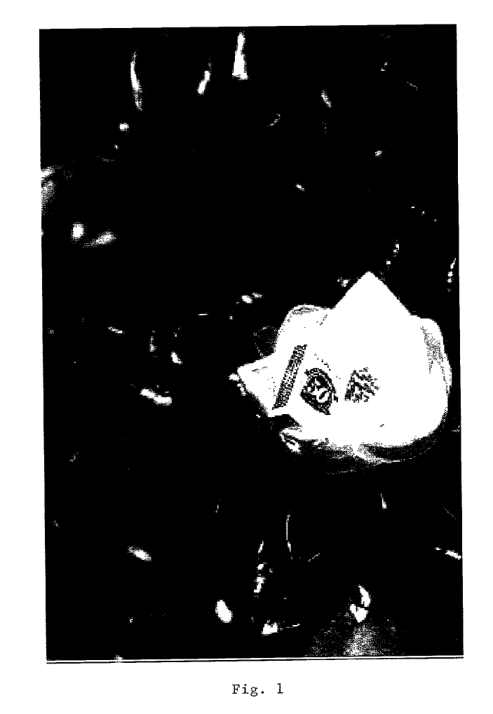

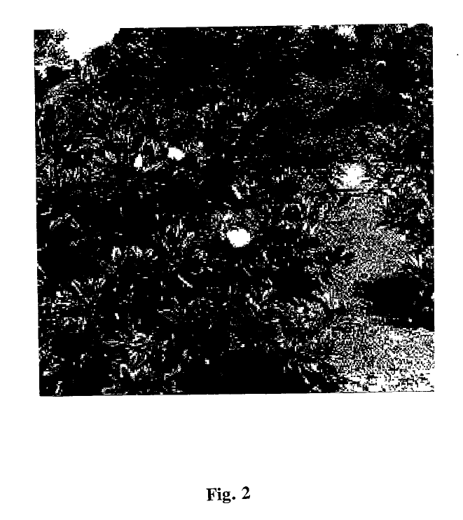

The accompanying color photographs show typical flowers and foliage of ‘STRgra’, with colors being as true as possible with illustrations of this type. FIG. 1 shows a close-up of the flower and foliage of ‘STRgra’. FIG. 2 shows the plant in its entirety.

### DETAILED BOTANICAL DESCRIPTION

Color references are made to The Royal Horticultural Society Colour Chart (RHS) except where general colors of ordinary significance are used. Colors are approximate as color depends on horticultural practices such as light level and fertilization rate, among other things.

Origin: Seedling from breeding program

PARENTAGE:

Male Parent.—

Female Parent.—Unnamed plant of Magnolia grandiflora

Classification:

Botanical.—Magnolia grandiflora

Commercial.—Magnolia cv. ‘STRgra’

PROPAGATION: Plants have been selected from the original seedling and propagated vegetatively by grafting in Monbulk, VIC, Australia

PLANT: Growth rate: Slow growth rate. From vegetative propagation flowering immediately. Flowering Season: Flowering from Spring to Autumn in subtropics; summer to Autumn in temperate climate.

Density.—Thick.

Growth Type.—Bush.

Height.—2000-2500 mm in 10 years

Width.—2000-2500 mm in 10 years

Stem Diameter.—4-6 mm

LEAF:

Color.—Deep green on top (glossy) RHS 147A, underside of leaf is rust-brown tomentose, RHS 152A

Length.—150-170 mm.

Width.—65-70 mm.

Apex.—Acute, cuneale.

Margin.—Entire, slightly recurved.

Mid Rib.—Cream RHS 144C, prominently raised below dark green RHS 137A.

Texture.—Glabrous and lustrous, glossy, hairy.

Shape.—Alternate, elliptical.

FLOWER:

Length of Flowering Period.—Long, 6 months (depending on climate)

Shape.—Cup-shaped.

Flower profile upper.—Cup shaped, upper wider than lower.

Flower profile lower.—Cup shaped, narrowing at base.

Number of flowers.—Many, solitary.

Lastingness of Bloom.—Flowers last approximately 3 days.

Color midzone outside.—Creamy-white RHS 155A.

Midzone inside.—Creamy-white RHS 155A Margin inside: Creamy-white RHS 155A.

Buds.—Bud shape: Ovate, spear-shaped Length: 70 mm Width: 30 mm Color: Flower buds encased in hairy pods which split to open, creamy-white RHS 155A

Petals:

Number of petals.—6-9 .

Size.—Very large.

Width.—80 mm.

Arrangement.—Overlapping.

Pedicels:

Pedicel prickles.—None.

Pedicel Length.—55 mm.

Sepals:

Sepal extensions.—Weak.

Sepal Length.—30 mm.

Basal spot: None

Filament color: Limey-cream RHS 1B

Anther color: Limey-cream RHS 1B

Style color.—Cream RHS 155B.

Pistils.—Many in an erect cone, color limey-cream RHS 1B.

Seed Vessel Characteristics:

Size.—Medium.

Color.—Pale green RHS 145B.

Fruit: Terminal cone approximately one or seeded carpets; scarlet RHS 60B when ripe

Fragrance of bloom: Typical fragrance of Magnolia grandiflora but stronger, sweet lemony scent

I Claim:

1. A new and distinct Magnolia plant named ‘STRgra’, as illustrated as described.
